(19) United States
(12) Patent Application Publication
(10) Pub. No.: US 2005/0288727 A1
(43) Pub. Date: Dec. 29, 2005

(54) Wireless sensing devices for evaluating heart performance

(76) Inventor: Abraham Penner, Tel Aviv (IL)

Correspondence Address: Bingham McCutchen, LLP, Suite 1800, Three Embarcadero, San Francisco, CA, 94111-4067, US

(21) Appl. No.: 11/140,267

(22) Filed: May 26, 2005

Related U.S. Application Data
(60) Provisional application No. 60/576,145, filed on Jun. 1, 2004.

(51) Int. Cl. A61N 1/362

(52) U.S. Cl. 607/32; 607/9; 600/509

(57) ABSTRACT

A system for monitoring heart performance comprises a plurality of sensing devices configured to attach to a patient's heart tissue and a controller. Each sensing device comprises a sensor configured to detect physiological data relating to heart contractility and a wireless transmitter configured to transmit data detected by the sensor. The controller comprises a receiver configured to receive the detected data transmitted by the plurality of sensing devices and a processor configured to analyze the received data.

### RELATED APPLICATION DATA

This application claims the benefit under 35 U.S.C. §119 of U.S. provisional application Ser. No. 60/576,145, filed Jun. 1, 2004.

### FIELD OF INVENTION

The present invention generally relates to the field of medical devices, and more specifically, to the use of wireless sensing devices for evaluating the performance and status of a heart muscle.

### BACKGROUND

A heart attack occurs when the blood supply to part of the heart muscle is severely reduced or stopped. The reduction or stoppage happens when one or more of the coronary arteries supplying blood to the heart muscle are blocked. Cardiac ischemia is a condition associated with lack of blood flow and oxygen to the heart muscle. As a result of the reduced blood flow, muscle cells at the heart may suffer permanent injury and may die.

While the heart contracts (during systole), the ventricle does not contract in a linear fashion. For example, part of the ventricle shortens relatively more in one direction or in a radial fashion. The change in the shape of the ventricle is progressive along its length and involves a twisting effect that tends to squeeze out more blood. If blood flow is cut or reduced to part of the heart muscle, myocardial infraction may occur. A few minutes after the blood flow is cut or reduced, damage to the heart may result, and the optimal contraction pattern of the heart may change. If the blood flow is resumed within hours from the onset of the cardiac ischemia, the heart muscle damage can be minimized, and in some cases, even reversed.

A person may have ischemic episodes without knowing it. For example, such individual may have painless ischemia called silent ischemia, which may deteriorate to a heart attack with no prior warning. A person with angina also may have undiagnosed episodes of silent ischemia. The diagnosis of ischemia is done mainly using non-invasive means, including an exercise test, a 24-hour portable monitor of an electrocardiogram (Holter monitor), echocardiogram, and stress echocardiogram.

In order to minimize damage associated with ischemia, early detection of ischemia or detection of its manifestations is desired. However, currently available techniques may not be able to detect ischemia and its manifestations, thereby failing to provide warning to a patient. For example, a stress test, such as a stress echocardiography (stress echo), is frequently used to evaluate heart performance or to detect a heart condition (e.g., coronary heart disease). Stress echo is an echocardiogram done, before and during, or immediately after, some form of physical stress (e.g., created by riding a bicycle or performing a treadmill exercise). This requires a physical effort from the patient, as well as special equipment and an echocardiography specialist, which increase test complexity and price, thereby limiting the use of the stress test to only cases with high risk of heart pathology.

### SUMMARY OF THE INVENTION

In one embodiment, a system for monitoring heart performance comprises a plurality of sensing devices configured to attach to a patient's heart tissue and a controller. Each sensing device comprises a sensor configured to detect physiological data relating to heart contractility and a wireless transmitter configured to transmit data detected by the sensor. The controller comprises a receiver configured to receive the detected data transmitted by the plurality of sensing devices and a processor configured to analyze the received data.

In another embodiment, a method for evaluating heart performance, comprises attaching a plurality of sensing devices to a patient's heart tissue, detecting, with the sensing devices, physiological data relating to heart contractility, wirelessly transmitting the detected data from the sensing devices to a controller, and analyzing the detected data at the controller to determine a contractility of the patient's heart.

In yet another embodiment, a system for monitoring heart performance comprises a plurality of sensing devices configured to attach to a patient's heart tissue, a controller, and a therapeutic medical device in which the controller is incorporated. Each sensing device comprises a sensor configured to detect physiological data relating to heart contractility and a wireless transmitter configured to transmit data detected by the sensor, wherein the sensing devices are configured to acoustically transmit the detected data to the controller. The controller comprises a receiver configured to receive the detected data transmitted by the plurality of sensing devices and a processor configured to analyze the received data.

### BRIEF DESCRIPTION OF THE DRAWINGS

In order to better understand and appreciate the invention, reference should be made to the drawings and accompany detailed description, which illustrate and describe exemplary embodiments thereof. For ease in illustration and understanding, similar elements in the different illustrated embodiments are referred to by common reference numerals. In particular:

FIG. 1 is a cutaway perspective view of a heart with attached sensing devices in accordance with one embodiment;

FIG. 2 is a perspective view of a heart with attached sensing devices in accordance with another embodiment;

FIG. 3 is a cutaway perspective view of a heart with attached sensing devices in accordance with yet another embodiment;

FIG. 4 is a schematic diagram of a system for monitoring heart performance constructed in accordance with still another embodiment;

FIG. 5 is a schematic diagram of a system for monitoring heart performance constructed in accordance with a still further embodiment of the present invention;

FIG. 6 is a schematic diagram of a system for monitoring heart performance constructed in accordance with yet another embodiment;

FIG. 7 is a flow chart of a method for monitoring heart performance in accordance with still another embodiment;

FIG. 8 is a flow chart of a method for monitoring heart performance in accordance with a further embodiment; and

FIG. 9 is a cutaway perspective view of a patient implanted with a system for monitoring heart performance in accordance with a still further embodiment.

### DETAILED DESCRIPTION OF THE ILLUSTRATED EMBODIMENTS

In the following description of the illustrated embodiments, it will be understood by those skilled in the art that the drawings and specific components thereof are not necessarily to scale, and that various structural changes may be made without departing from the scope or nature of the various embodiments.

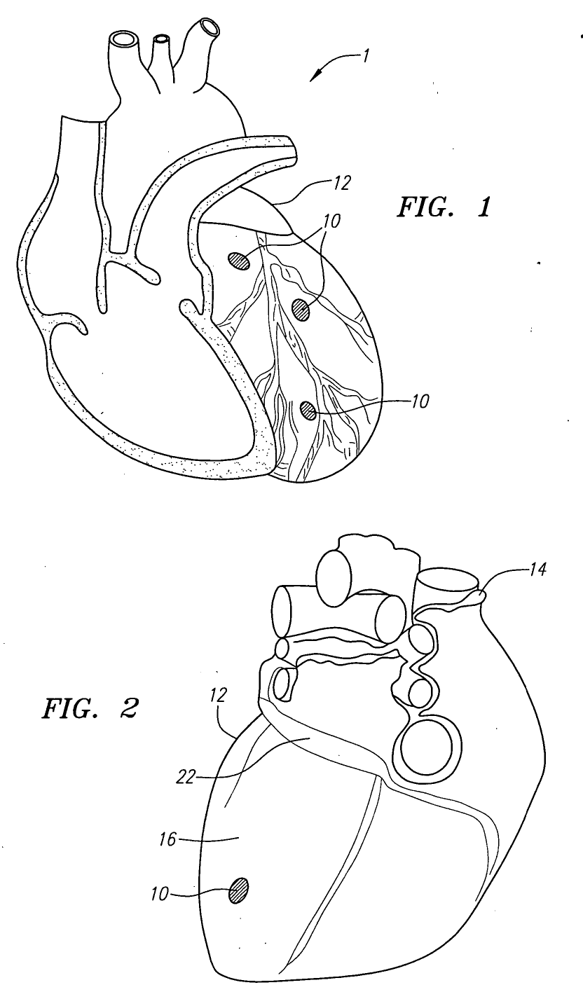

As illustrated in FIG. 1, in accordance with some embodiments of the invention, a system 1 includes a plurality of sensing devices 10 configured to be attached to a heart 12. Each sensing device 10 includes a sensor 11 and a wireless communication device 32. The sensing devices 10 are configured to measure a characteristic of the heart 12, such as its contractility, or a variable associated with contractility of the heart 12. From that measured characteristic, the system 1 can determine a performance of the heart 12. As used herein, the words “heart tissue” refer to myocardium and pericardium 14.

Various types of sensors can be used to sense one or more parameters associated with a heart condition, such as parameters that can be used as indicators for ischemia.

In some embodiments, position sensors 11 sense locations or orientations of portions of a heart 12. The sensed locations or orientations can be used to extrapolate contractility of the heart 12. Changes in the sensed locations or the sensed orientations can also be used to extrapolate contractility of the heart 12. In some embodiments, the determined locations or orientations can be combined using an algorithm to form a three dimensional time dependent map of the heart 12. In some embodiments, sensors 11 use magnetic fields to determine locations or orientations. In other embodiments, radio-opaque positioning sensing devices 10 are used to determine locations or orientations. In other embodiments, triangulation is used to determine the locations of sensing devices 10.

In some embodiments, a sensor's velocity is calculated by taking a first derivative of the sensor's position over time. The determined velocity is used to determine the contractility of a heart 12. In other embodiments, a sensor's acceleration is calculated by taking a second derivative of the sensor's position or a first derivative of the velocity over time. The determined acceleration is used to determine the contractility of the heart 12.

In other embodiments, the sensors 11 are accelerometers for measuring accelerations of portions of a heart 12. A variety of accelerometers can be used. For example, accelerometers integrated within pacemakers can be used. MEMS technology can be employed to reduce a size of the accelerator, thereby reducing a size of the sensing devices 10. The accelerations or changes of the accelerations of the portions of the heart 12 are then used to determine the contractility of the heart 12. In some embodiments, signals from accelerometer sensing devices 10 are integrated over time to obtain velocities, which are used to determine the contractility of the heart 12. In other embodiments, the velocities are integrated over time to obtain distances, which are also used to determine the contractility of the heart 12.

In other embodiments, the sensors 11 detect velocities of portions of a heart 12. The velocities or changes of the sensed velocities can be used to determine the contractility of the heart 12.

In other embodiments, the sensors 11 are strain gauges configured to monitor strains on portions of a heart 12 as it contracts. The detected strains or changes of the detected strains are used to determine the contractility of the heart 12. In some embodiments, the sensors 11 are configured to detect a change, in response to damage to the heart 12, of the strain induced by contraction of the heart 12.

In other embodiments, the sensors 11 are tactile sensors for detecting changes in the stiffness of a heart 12. Stiffness of the heart 12 can change due to contraction and relaxation of the heart 12, or due to ischemic damage to the heart 12 from myocardial infractions. The detected heart stiffness or change thereof can be used to determine the contractility of the heart 12, or to monitor the heart diastolic filling.

Also in other embodiments, sensors 11 are configured to detect an electrical impedance of a heart 12. As cells die, the their electrical impedance changes. As such, by monitoring an electrical impedance of a portion of the heart 12, the vitality of the cells in the portion of the heart 12 can be determined. In still other embodiments, sensors 11 are configured to detect electrical activity in a portion of a heart 12, as in an electrocardiogram. In other embodiments, sensors 11 are configured to detect the temperature of a portion of a heart.

Sensing devices 10 can communicate in various ways with controllers 13 incorporated in other implantable devices 28 or external devices 26. Controllers can also be incorporated in therapeutic medical devices or diagnostic medical devices. Diagnostic medical devices include devices for displaying an image of the heart to a physician in a well known fashion. In some embodiments, a wireless communication device 32 sends signals from and receives signals sent to the sensing devices 10. The wireless communication device 32 can send and receive, an acoustic signal, a magnetic induction signal, an optical signal (e.g., UV, infrared), or an electromagnetic signal (e.g., a radio-frequency signal) to and from the sensing devices 10. In other embodiments, the communication can be performed using a conventional wire lead 30.

Examples of implantable devices 28 include pacemakers, defibrillators, implantable cardioverter defibrillators, cardiac resynchronization therapy (CRT) pacemakers, CRT-defibrillators, and nerve stimulators. Examples of external devices 26 include external pulse generators and telemetry recording devices.

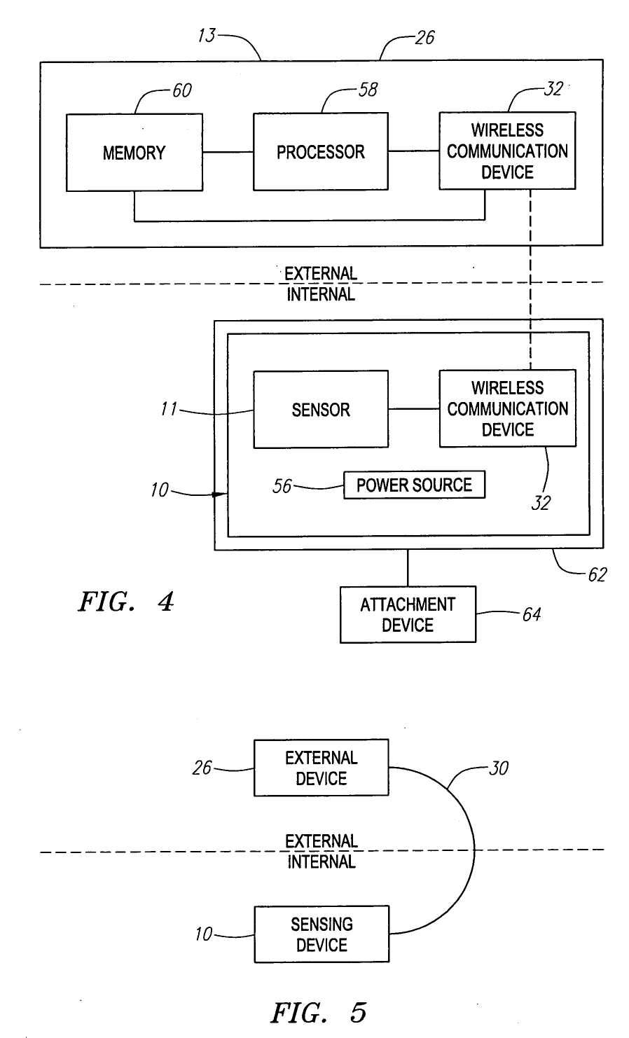

In some embodiments, as shown in FIG. 4, the controller 13 also has a wireless communication device 32 for receiving signals from and sending signals to the sensing devices 10. In some embodiments, the wireless communication devices 32 in the system 1 are transceivers and the respective controller 13 and sensing devices 10 for an acoustic communication network. In still other embodiments, the wireless communication devices 32 in the sensing devices 10 are configured to convert acoustic energy transmitted by the wireless communication devices 32 in the controller 13 into electrical energy used to operate the respective sensing devices 10.

The system 1 also includes a power source 56 for the sensing devices 10. The power source 56 can be one or more internal batteries. Alternatively, the sensing devices 10 can be powered telemetrically using energy from radio frequency, acoustic, magnetic or infrared signals.

In some embodiments, the system 1 also includes a processor 58 for processing signals from the sensing devices 10. The processor 58 of some embodiments is disposed in the external device 26, but in alternative embodiments, the processor 58 can be disposed in the sensing devices 10. In still other embodiments, the processor 58 can be disposed both in the external device 26 and in the sensing devices 10. In some embodiments, the system 1 also include a memory 60 for storing the data from the sensor and the processed data.

In some embodiments, the system 1 includes an encapsulation 62 for the sensing devices 10 and wireless communication device 32 for improving a durability of those implanted parts. The system 1 also includes attachment devices 64 for attaching the sensing devices 10 to the heart. Suitable attachment devices 64 include screws, hooks, sutures, anchors, suction devices, and clips.

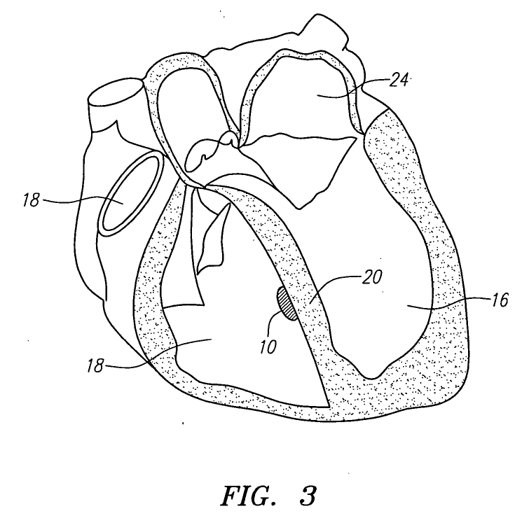

In some embodiments, the system 1 also includes a delivering device for delivering the sensing devices 10 to target sites. Suitable delivery devices include catheters, injection needles, and cannulas. The sensing devices 10 can be attached to the pericardium 14 of the heart 12, and preferably over the left ventricle 16, as shown in FIG. 2. However, the sensing devices 10 can also be attached to other locations on the heart 12. Various techniques can be used to attach the sensing devices 10 to the heart 12. For examples, the sensing devices 10 can be implanted, sutured, or attached to the heart during a heart surgery, such as a coronary artery bypass surgery (CABG) or a valve replacement. This surgery can be a conventional one with incision of the sternum or a minimally invasive one, which is performed through a smaller incision on the patient's chest over the heart to gain access to the coronary arteries.

Alternatively, the sensing devices 10 can be implanted percutaneously in the right heart chambers 18, preferably in the septum 20, as shown in FIG. 3, or in the coronary sinus 22. In other embodiments, the sensing devices 10 can be implanted using a trans-septal approach in the left atrium 24 or the left ventricle 16. In other embodiments, the sensing devices 10 can be secured to other parts of the heart 12 by other conventional methods.

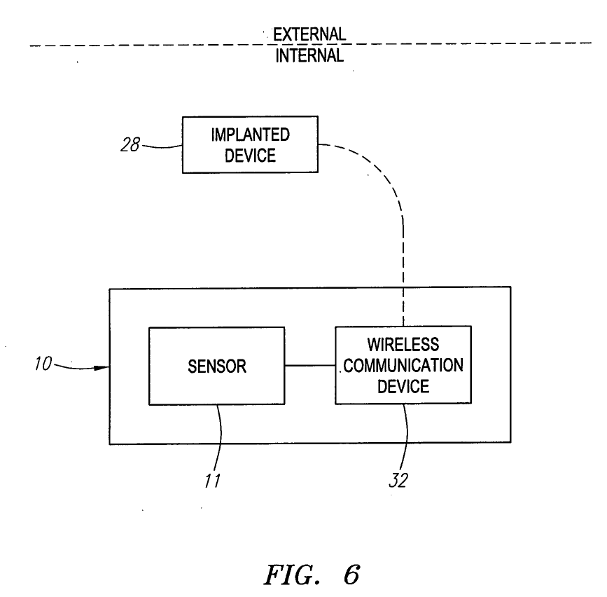

In some embodiments, as shown schematically in FIGS. 4 and 5, the sensing devices 10 are configured to communicate with an external device 26. In other embodiments, as shown schematically in FIG. 6, the sensing devices 10 are configured to communicate with an implanted device 28 internal to a patient's body, such as an implantable pulse generator. The communication can be accomplished using conventional leads 30, as shown in FIG. 5, or a wireless communication device 32, as shown in FIG. 4. Wireless communication devices 32 include transmitters, receivers, and transceivers.

In case of ischemia, parts of the heart muscle 12 that have a reduced blood supply lose part of their ability to contract and relax after a contraction. The sensing devices 10 may be used to detect ischemia by monitoring the heart contractility or an abnormality or a change in the heart tissue movement. These changes can occur at the stage of relaxation after systole or during a contraction at the systolic phase. During ischemia, the sensing devices 10 attached to the heart 12 senses a characteristic (e.g., a contractility, or a variable associated with a contractility) of the heart 12 that is associated with a symptom of ischemia. Based on the sensed characteristic, a heart condition (e.g., existence of a blockage of artery, severity of the stenosis, etc.) can be determined. Based on the determined heart condition, a physician can determine the patient status, perform additional examinations, or provide an appropriate treatment (i.e. catheterization, drug therapy etc.).

In other embodiments, the sensing devices 10 can be used for evaluating a status of congestive heart failure (CHF) patients. Heart failure is generally divided into systolic and diastolic. In systolic heart failure, the heart or parts of it lose the ability to contract. Diastolic dysfunction caused by abnormalities in left ventricular filling can be a result of many pathologic conditions, including hypertrophy, infiltrative cardiomyopathies, or myocardial ischemia. Attaching sensing devices 10 to the heart 12, and especially to the left ventricle 16, as shown in FIGS. 1 and 2, can help in evaluating the status of the patient. This is true for both systolic dysfunction where the contractility can be monitored and for diastolic dysfunction where the relaxation and filling of the heart 12 can be followed.

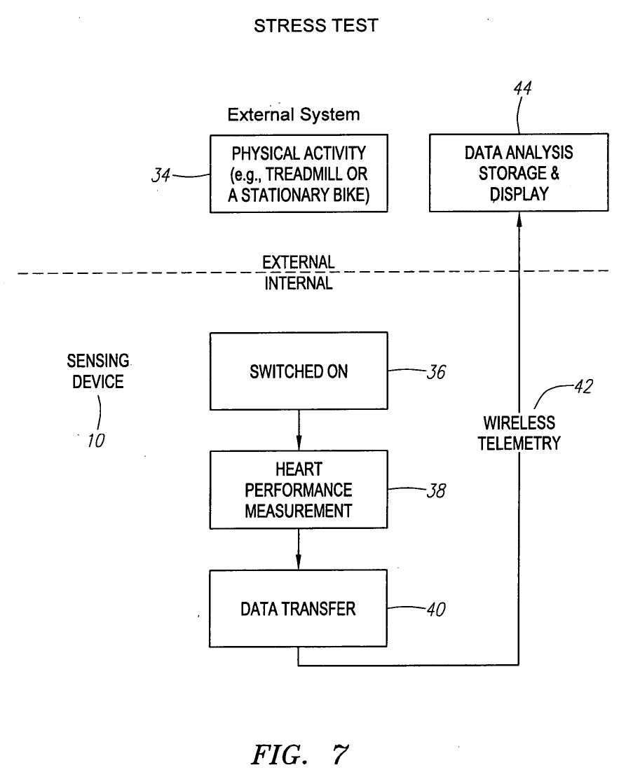

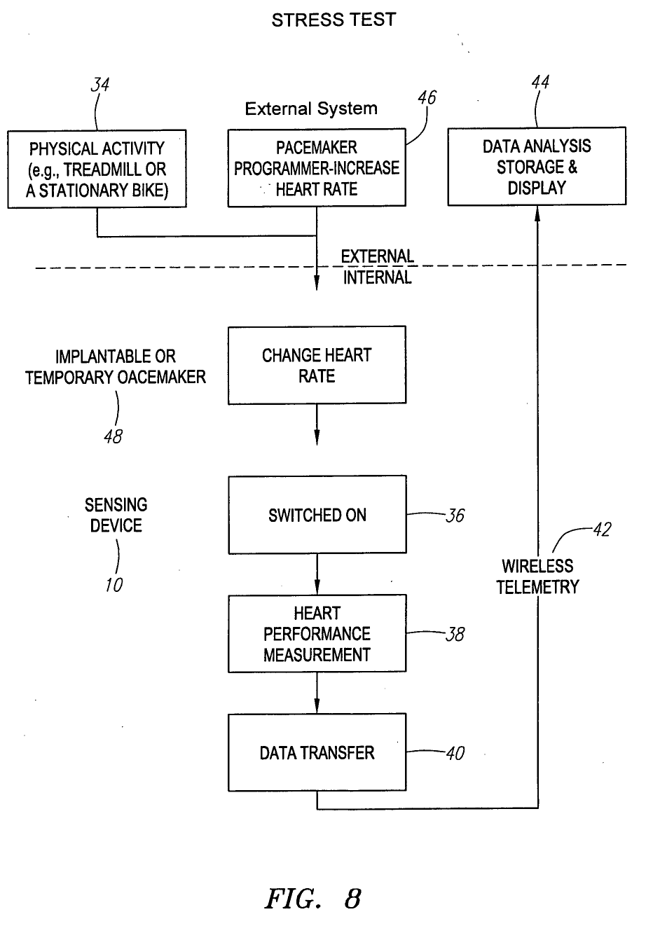

In other embodiments, the sensing devices 10 can be used to monitor heart performance under a stress test, as shown in FIG. 7. A stress test involves performing a simple exercise (usually a treadmill or a stationary bike) while the patient is monitored using several devices. These devices may include an electrocardiograph machine (ECG), an ultrasound machine, a blood pressure cuff, and/or a mask.

As shown in FIG. 7, the process begins with the start of physical activity 34 and activation of the sensor 36. Next, a heart characteristic associated with contractility is measured 38. Then data is transferred 40, via wireless telemetry 42, to an external system, where it is analyzed, stored, and displayed 44. In other embodiments, based on a measured heart characteristic (e.g., contractility or a variable associated with a contractility), a map of heart movement can be formed.

For a patient with an implantable pacemaker, the heart rate can be increased by increasing the electrical stimulation rate of the pacemaker with no physical activity by the patient, as shown in FIG. 8. This method is similar to that depicted in FIG. 7, except the test may also begin by increasing the electrical stimulation rate of the pacemaker 46. This allows the physician to carry out the “stress test” at any location, such as the clinic, office, or the patient's home. In some cases, the stress test can be performed by remote programming of the pacemaker using a telemetric system such as the Medtronic CareLink™.

In another embodiment sensing devices 10 are used to monitor heart performance under a stress test involving a temporary pacemaker 48. The temporary pacemaker 48 may be used to make a heart 12 beat at a normal rate after heart surgery or another life-threatening event involving the heart 12. The temporary pacemaker 48 can be external or internal to the patient's body. Using the above-described method, a heart stress test can be performed while the patient is recovering from the heart surgery. In such cases, the sensors 11 sense a characteristic of the heart 12 (e.g., contractility or a variable associated with a contractility) and transmit a signal providing feedback to the physician.

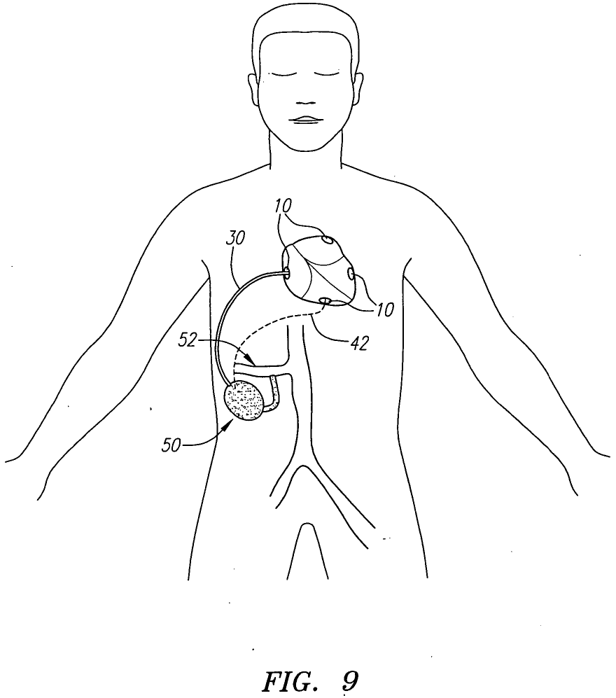

In other embodiments, the sensing devices 10 can be used to automatically perform a heart test and use the test results to optimize an operation of a therapeutic device, such as an implantable pulse generator. Another embodiment is described in FIG. 9. The sensing devices 10 on the heart 12 are used for feed back regulation of a drug pump 50. The sensors 11 can be of any type disclosed herein. For example, the sensors 11 can be an accelerometer, a velocity sensor, a position sensor, a tactile sensor, or a pressure sensor.

As shown in the illustrated embodiment, the sensing devices 10 are configured to communicate with a drug pump 50 using a conventional lead 30 or a wireless communicator 42. Based on data from the sensor devices 10, the drug pump 50 can control a dosage of medication, and optimize an amount of medication injected to the patient via an injection port 52. In other embodiments, the communication between the sensing devices 10 and the drug pump 50 can be performed indirectly via another implantable device (not shown) such as a pacemaker, a pacemaker, an implantable cardioverter defibrillator, a cardiac resynchronization therapy (CRT) pacemaker, a CRT-defibrillator, or a nerve stimulator.

In other embodiments, heart muscle movement can be used for optimizing a CRT operation. Sensing devices 10 can be implanted in the heart wall and septum 20 to detect movement, which can then be used to optimize the bi-ventricular delay of CRT. The optimization can be done by transferring the information to an external system and then reprogramming the CRT, or by an automatic feedback of the CRT operation using the measurements from the sensing devices 10. For patients with pacemakers, the system can be used for feedback regulation of the pacemaker to control the pace and rate of a heart based in part of the measured heart characteristic.

Although various embodiments of the invention have been shown and described herein, it should be understood that the above description and figures are for purposes of illustration only, and are not intended to be limiting of the invention, which is defined only by the appended claims and their equivalents.

1. A system for monitoring heart performance, comprising:
a plurality of sensing devices configured to attach to a patient's heart tissue, each sensing device comprising
a sensor configured to detect physiological data relating to heart contractility, and
a wireless transmitter configured to transmit data detected by the sensor; and
a controller comprising
a receiver configured to receive the detected data transmitted by the plurality of sensing devices, and
a processor configured to analyze the received data.

2. The system of claim 1, wherein at least one of the sensing devices is configured to attach to heart tissue located on an exterior of a heart.

3. The system of claim 1, wherein at least one of the sensing devices is configured to attach to heart tissue located on an interior of a heart.

4. The system of claim 1, wherein the controller is incorporated in a device configured for implantation in the patient.

5. The system of claim 1, wherein the controller is incorporated in a device configured for use external to the patient.

6. The system of claim 1, wherein the controller is incorporated in a therapeutic medical device.

7. The system of claim 1, wherein the controller is incorporated in a diagnostic medical device.

8. The system of claim 1, wherein the data is selected from the group consisting of position, velocity, acceleration, change in position, change in velocity, change in acceleration, stiffness, strain, electrical impedance, temperature, and electrical activity.

9. The system of claim 1, wherein the respective sensing device sensors are selected from the group consisting of position sensors, velocity sensors, accelerator sensors, strain sensors, tactile tensors, temperature sensors, electrocardiogram monitors, and electrical impedance sensors.

10. The system of claim 1, wherein the sensing devices acoustically transmit the detected data to the controller.

11. The system of claim 1, wherein the sensing devices transmit the detected data to the controller using a signal selected from the group consisting of acoustic, radio frequency, magnetic induction, and infrared.

12. The system of claim 6, wherein the therapeutic device comprises an implantable pulse generator selected from the group consisting of a pacemaker, a defibrillator, an implantable cardioverter defribrillator, a CRT-pacemaker, a CRT-defibrillator, and a nerve stimulator.

13. The system of claim 1, wherein the controller is incorporated in, or coupled with, an external pulse generator.

14. The system of claim 6, wherein the detected data is used for controlling an output of the therapeutic medical device.

15. The system of claim 14, wherein the medical device comprises a pump that delivers a therapeutic agent to the patient.

16. The system of claim 1, wherein the processor is configured to analyze the detected data to determine a contractility of the patient's heart.

17. The system of claim 1, wherein the sensing device transmitters comprise transceivers, the controller receiver comprises a transceiver, and the respective controller and sensing device transceivers comprise an acoustic communication network.

18. The system of claim 17, wherein the sensing device transceivers are configured to convert acoustic energy transmitted by the controller transceiver into electrical energy.

19. A method for evaluating heart performance, comprising:
attaching a plurality of sensing devices to a patient's heart tissue;
detecting, with the sensing devices, physiological data relating to heart contractility;
wirelessly transmitting the detected data from the sensing devices to a controller; and
analyzing the detected data at the controller to determine a contractility of the patient's heart.

20. The method of claim 19, wherein attaching a plurality of sensing devices to a patient's heart tissue comprises attaching at least one of the sensing devices to heart tissue located on an exterior surface of the patient's heart.

21. The method of claim 19, wherein attaching a plurality of sensing devices to a patient's heart tissue comprises attaching at least one of the sensing devices to heart tissue located on an interior surface of the patient's heart.

22. The method of claim 19, wherein the data is selected from the group consisting of position, velocity, acceleration, changes in position, changes in velocity, changes in acceleration, stiffness, strain, electrical impedance, temperature, and electrical activity.

23. The method of claim 19, wherein wirelessly transmitting the detected data comprises acoustically transmitting the detected data.

24. The method of claim 19, wherein wirelessly transmitting the detected data comprises using a signal selected from the group consisting of acoustic, radio frequency, magnetic induction, and infrared.

25. The method of claim 19, further comprising controlling an output of a therapeutic medical device based, at least in part, on the determined contractility.

26. The method of claim 19, further comprising controlling a pacing signal used to pace the patient's heart based, at least in part, on the determined contractility.

27. The method of claim 19, further comprising transmitting an acoustic signal from the controller to the plurality of sensing devices in order to activate the respective sensing device sensors.

28. The method of claim 27, further comprising converting, at the sensing devices, the acoustic signal into electrical energy.

29. The method of claim 19, wherein the controller is incorporated in a device configured for implantation in the patient.

30. The method of claim 19, wherein the controller is incorporated in a device configured for use external to the patient.

31. The method of claim 19, wherein the controller is incorporated in a therapeutic medical device.

32. The method of claim 19, wherein the controller is incorporated in a diagnostic medical device.

33. A system for monitoring heart performance, comprising:
a plurality of sensing devices configured to attach to a patient's heart tissue, each sensing device comprising
a sensor configured to detect physiological data relating to heart contractility, and
a wireless transmitter configured to acoustically transmit data detected by the sensor;
a controller incorporated in a diagnostic or therapeutic medical device comprising
a receiver configured to receive the detected data transmitted by the plurality of sensing devices, and
a processor configured to analyze the received data.
